(19) United States
(12) Patent Application Publication
(10) Pub. No.: US 2003/0214629 A1
(43) Pub. Date: Nov. 20, 2003

(54) Wide field of view lens

(76) Inventors: K. Peter Luloh, Longwood, FL (US); Michael Annen, Sanford, FL (US)

Correspondence Address: BEUSSE, BROWNLEE, BOWDOIN & WOLTER, P. A., 390 NORTH ORANGE AVENUE, SUITE 2500, ORLANDO, FL, 32801, US

(21) Appl. No.: 10/393,042

(22) Filed: Mar. 20, 2003

Related U.S. Application Data
(60) Provisional application No. 60/380,906, filed on May 15, 2002.

(51) Int. Cl. A61B 3/00

(52) U.S. Cl. 351/200

(57) ABSTRACT

A lens system and method for use in eye surgery and diagnostics in combination with a microscope is provided. The lens system includes a lens having a variable field curvature, so that the concave surface of a fundus of the eye is in focus for substantilly the full extent of a retina. The lens system also provides a lens adapted to compensate for optical properties of eye components in an image space. A method for designing a wide angle lens system for use in combination with a microscope for eye surgery includes defining a number of eye components having optical properties as surfaces in the lens system and calculating a wide angle lens design based on a desired field of view and the surfaces defined from the plurality of eye features modeled as optical components.

### CROSS REFERENCE TO RELATED APPLICATIONS

This application claims priority to a provisional application filed on May 15, 2002 having application Ser. No. 60/380,906, the specification of which is incorporated herein by reference.

### FIELD OF THE INVENTION

The present invention is generally related to ophthalmic viewing systems, and, in particular, to a wide angle lens system for viewing the retina of an eye.

It is known to use binocular operating microscopes adapted to assist ophthalmologists in performing eye surgery. While providing magnification and viewing of small areas at the back of the eye, such as the retina, ophthalmic operating microscopes are typically limited to a minimum magnification and a maximum field of view that precludes viewing of the extreme edges of the retina. It is known to extend the field of view capability of such microscopes to enable complete examination of the entire retina by using devices appended to the microscope. For example, a device may consist of two parts: a lens placed in between the microscope and the patient's eye, and an optical system placed within the microscope that corrects for an inherent inversion of the image produced by the first part. One such device is described in U.S. Pat. No. 6,212,006 and marketed by Oculus Optikgeraete GmBH.

The wide angle system described in the '006 patent uses a single glass lens to provide a wide angle view of the retina. However, the image produced by such a system may not be sufficiently sharp to allow an ophthalmologist to view physiological detail at the edge of the retina during an eye examination. While wide angle lenses used in these applications are typically optimized for image quality, the optimization involves reproducing, through variation of the lens material, thickness, and surface curvatures, the characteristics present in a flat object onto a flat image plane. However, in the case of the fundus of an eye, the desired object to be viewed is not flat. Rather, the fundus, including the retina, is concave in shape. Usually in a lens design, it is a problem when a curved object surface images to a flat image surface, and the image needs to be corrected by choosing appropriate lens curves, thicknesses, and materials. As a result, attempts to optimize lenses based on viewing a flat object will necessarily result in aberrations of the image at the extremities of the field of view. In addition, the eye is composed of many optical elements between the cornea and the retina, that affect light transmission through the eye and that need to be considered in the calculation of the image quality of viewing optics. Significantly, the cornea, the crystalline lens, the vitreous humor, and the aqueous humor act as additional optical components in the light path of any retina viewing instruments.

Thus, it would be desirable to provide system and method for viewing the entire retina that is not subject to the foregoing drawbacks. That is, it would be desirable to provide an optimized lens system and method of designing a lens that is not subject to substantial aberration of retinal images at the periphery of the retina when viewing the interior of an eye. In addition, it would be desirable to provide a lens that has a field curvature equal to the retina curvature. Further, it would be desirable to provide an optimized lens system and method of designing a lens that considers the optical elements of the eye and the effect that these elements have on light transmission through the eye.

### SUMMARY OF THE INVENTION

Generally, the present invention fulfills the foregoing needs by providing, in one aspect, a lens system for use in eye surgery and diagnostics, in combination with a microscope. The system includes a lens having a configuration for providing variable field curvature, wherein the concave surface of a fundus of the eye is in focus for at least the full extent of a retina of the eye. The system also includes a lens adapted to compensate for optical properties of eye components in an image space.

The present invention further fulfils the foregoing needs by providing, in another aspect thereof, a method of designing a wide angle lens system for use in combination with a microscope for eye surgery and diagnostics. The method includes defining a plurality of eye components having optical properties as surfaces in the lens system. The method also includes calculating a wide angle lens design based on a desired field of view and the surfaces defined from the plurality of eye features modeled as optical components. The method further includes determining a desired wide field of view by providing a field curvature to the lens so that the concave surface of a fundus of an eye is in focus for at least the full extent of a retina of the eye.

### BRIEF DESCRIPTION OF THE DRAWINGS

The features and advantages of the present invention will become apparent from the following detailed description of the invention when read with the accompanying drawings in which:

FIG. 1 illustrates a sectional view of the lens system of the invention including a wide angle lens, an eye, and a viewing microscope.

FIG. 2 illustrates an optical layout showing light rays traveling through a model eye and an exemplary wide angle lens.

### DETAILED DESCRIPTION OF THE INVENTION

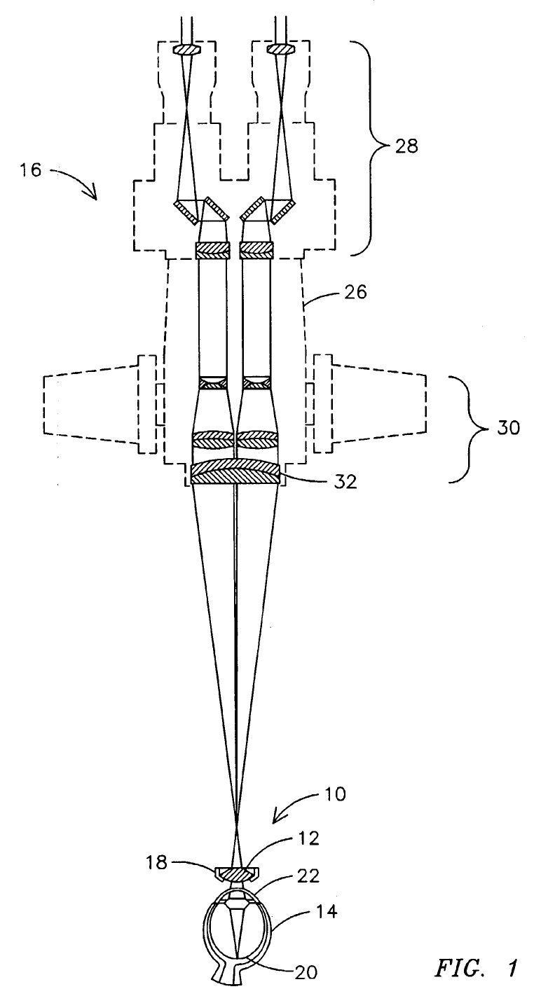

FIG. 1 illustrates a sectional view of the lens system 10 of the invention including a wide angle lens 12, an eye 14, and a viewing microscope 16. Generally, the lens system 10 includes a wide angle lens 12 designed for viewing the entire retina 20 of the eye 14 and a lens mount 18 for retaining the lens 12. the lens mount 18 allows positioning of the lens 12 between the eye 14 of a patient and the microscope 16 to allow viewing of the retina 20. In an aspect of the invention, the lens 12 can be adapted to be positioned in contact with the cornea 22 of the eye 14. As known, the microscope 16 (enclosed in a housing 26, schematically illustrated) includes an eyepiece arrangement 28, a magnification changer 30, and a lens arrangement 32 to magnify and direct an image to a viewer (not shown). P In use, an ophthalmic surgeon selects a lens 12 to provide a wide angle view of a patient's retina 20, for example, during surgery or examination. In an aspect of the invention, the lens can be formed from a disposable polymer material designed for a single use to eliminate the need for cleaning and disinfecting the lens after each use. After choosing a lens, the surgeon inserts the lens 12 into the lens mount 18 and positions the microscope 16 and lens mount 18 over the patient's eye 14. In an embodiment, the lens 12 position can be positioned to be in contact with the cornea 22 of the eye 14. The microscope 16 is than adjusted for focus and the lens mount 18 is repositioned with respect to the eye 14 to achieve a desired field of view over the extent of the retina 20.

Importantly, the inventors have innovatively realized that a lens 12 designed to focus on a curved surface (in contrast to a conventional wide angle retina viewing lens designed to focus on a flat surface) can provide an improved wide angle image of the retina 20. Accordingly, the lens 12 of the invention is optimized to focus on a curved, instead of a flat, surface. The field curvature of the lens can be configured to provide a variable back focal length which compensates for the curvature of the retina 20. Consequently, substantially the entire retina can be viewed with minimal distortion or aberration. In addition, the inventors have also realized that a wide angle lens for viewing the retina of an eye can be further-optimized by considering the effects of the optical elements within the eye 12, thereby achieving better resolution. As a result, an improved lens system, in conjunction with a microscope, is provided for viewing the full extent of the retina.

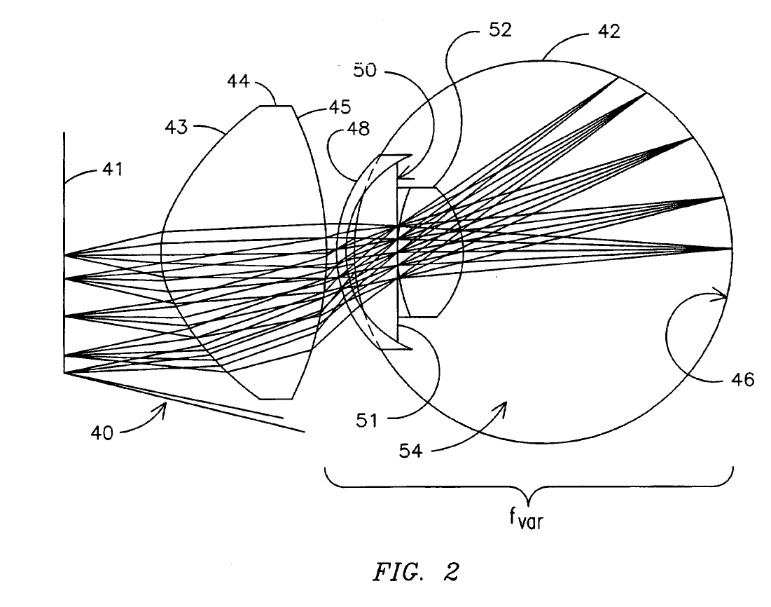

FIG. 2 illustrates an optical layout showing light rays 40 traveling through a model eye 42 and an exemplary wide angle lens 44. In the exemplary embodiment depicted in FIG. 2, the image for the lens system is formed on the retina 46. Accordingly, the lens 44 has a primary, or object surface 43 and a secondary, or image surface 45 as defined by designating the retina 46 as forming the image of the lens system. It should be understood that this imaging convention could be reversed to achieve equivalent results. In an embodiment, the lens 44 can be a two-sided convex lens having a positive refractive power, where one or more of lens surfaces is an even aspherical surface. By modeling the internal components of the eye 42 and using the modeled components in calculations to generate a lens design, an improved wide angle lens 44 can be created for use in ophthalmic surgery applications, such as the application described previously. In an aspect of the invention, the curvature of the retina 46 can be included in the lens design calculations to optimize the lens 44 for viewing the entire extent of the retina 46. For example, the lens 44 can be designed to provide a variable back focal length, fᵥₐᵣ, that compensates for the curvature of the retina 46 from the central axis of the model eye 42, so that the full extent of the retina 46 is in focus when viewed through the lens 44. Accordingly, a field curvature corresponding to a retinal curvature can be incorporated into the lens 44 design to ensure that the full extent of the retina 46 is in focus. As shown in FIG. 2, the eye model can include components such as a cornea 48, a crystalline lens 52, aqueous humor 50, and vitreous humor 54.

As shown, the light rays 40 travel from an object plane 41 of the microscope lens arrangement 32 and enter the lens 44 at the object surface 43. The light rays 40 are focused as they travel through the lens 44 and are further focused corresponding to a desired field curvature as they emerge at the image surface 45. The light rays then pass through the cornea 48 and aqueous humor 50 undergoing further refraction until reaching a primary vertex plane of the crystalline lens 51. The rays are focused in the lens 52 and pass through the vitreous humor 54 before forming a curvature-corrected image on the retina 46.

To further ensure that an accurate image of the retina 46 is formed by the wide angle lens 44, the lens design can include parameters to compensate for the optical properties of various eye components in an image space. Accordingly, each of the eye model 42 components can be mathematically characterized, and the characterizations can be included in the lens design calculation. For example, the eye model 42 components can be characterized by each component's respective surface, radius, thickness, aperture, diameter, and refractive properties. The characterizations can be included in the lens design calculation to compensate for the effects of each eye component in the image space of the eye model 42. As a result, a wide angle lens 44 can be created that is tuned for viewing the entire retina 46 through the lens 44, in conjunction with, for example, a microscope.

An example of a wide angle lens design adapted for viewing the entire extent of the retinal of an eye will now be described. As is known, lenses can be designed using “off the shelf” optical design software such as ZEMAX™, available from Focus Software, Incorporated and OSLO™, available from Lambda Research Corporation. By providing design parameters for a desired lens and the corresponding image and object spaces, the software programs calculate the desired lens configuration.

Generally, a method for designing a wide angle lens can include defining eye components modeled as optical components as surfaces in the image space of the lens, and then calculating a wide angle lens design based on a desired field of view and the surfaces defined from the optical components of the eye. The desired field of view can be established by providing a variable lens back focal length which compensates for the curvature of the retina from the central axis of the model eye, so that the concave surface of a fundus of the eye is in focus for at least the full extent of the retina of the eye. Accordingly, a wide angle lens design can be implemented by retrieving model eye information, such as the optical characteristics of the cornea, aqueous humor, crystalline lens and vitreous humor of the eye, available from known sources, such as the Indiana University School of Optometry. These characteristics are then interpreted and defined as appropriate parameters for inclusion in the lens design software, such as each eye component's respective surface, radius, thickness, aperture, diameter, and refractive properties.

Next, a prototype lens design can be chosen based on desired characteristics and known lens configurations. By using lens design software in an iterative process, the lens can be designed modeled and simulated to achieve desired focusing characteristics. For example, a list of desired image properties and their weighting for these properties can be compiled. This list is known in the art as the merit function of the lens. The merit function is input into the lens design software and the software iteratively makes a small change in values of the initial desired properties and then calculates whether the merit function is improved. If the merit function improves, the changes continue. If not, the changes are rejected. The process iterates until the user stops it. Thus, an appropriate lens design can be generated by the software program. As a result of using the modeled eye components and a field curvature corresponding to the curvature of the retina, an improved lens design optimized for wide angle viewing of the retina of the eye can be generated.

Tables 1-3 below depict input and output values for an exemplary field curvature configured lens design according to an aspect of the invention. Table 1 below depicts general lens data for the design, derived from optical eye model parameters and desired imaging characteristics including field curvature corresponding to the retina of the eye.

TABLE 1
GENERAL LENS DATA

| Surfaces | 13 |
|---|---|
| Stop | 8 |
| System Aperture | Float By Stop Size = 1.5 |
| Glass Catalogs | eye_best schott OLD_OHAR MISC |
| Ray Aiming | Real Reference, Cache On |
| X Pupil shift | 0 |
| Y Pupil shift | 0 |
| Z Pupil shift | 0 |
| Apodization | Uniform, factor = 0.00000E+000 |
| Effective Focal Length | 8.094134(in air) |
| Effective Focal Length | 10.81361(in image space) |
| Back Focal Length | −23.01264 |
| Total Track | 35.0804 |
| Image Space F/# | 0.626567 |
| Paraxial Working F/# | 5.124857 |
| Working F/# | 4.797881 |
| Image Space NA | 0.09730458 |
| Object Space NA | 0.2116116 |
| Stop Radius | −1.5 |
| Paraxial Image Height | 15.55141 |
| Paraxial Magnification | −2.173199 |
| Entrance Pupil Diameter | 12.91823 |
| Entrance Pupil Position | −35.62895 |
| Exit Pupil Diameter | 3.12316 |
| Exit Pupil Position | −20.3983 |
| Field Type | Real Image height |
| Maximum Field | 10.5 |
| Primary Wave | 0.5875618 |
| Lens Units | Millimeters |
| Angular Magnification | −3.132101 |

Table 2 depicts a summary of the surface data associated with each of the optical components in the lens system. As shown, the lens system includes 13 surfaces and includes the type, radius, thickness, material (Glass) diameter and conic corresponding to each surface. Notably, the retinal surface, IMA, is described here as being curved so that the resulting design, unlike conventional lens designs for flat retinal viewing, is optimized for wide angle focusing on the curvature of the retina.

TABLE 2
SURFACE DATA SUMMARY

| Sur- | | Radi- | | | | |
|---|---|---|---|---|---|---|
| face | Type | us | Thickness | Glass | Diameter | Conic |
| OBJ | STANDARD | Infin- | 5.796747 | n/a | 14.77025 | 0 |
| | | ity | | | | |
| 1 | EVENASPH | R1 | 10 | POLY- | 18 | C1 |
| | | | | STYR | | |
| 2 | EVENASPH | R2 | 0 | n/a | 18 | C2 |
| 3 | STANDARD | Infin- | 2 | n/a | 15.65935 | 0 |
| | | ity | | | | |
| 4 | STANDARD | 11.7 | −1 | n/a | 23.4 | 0 |
| 5 | EVENASPH | 7.8 | 0.55 | CORNEA | 12 | 0 |
| 6 | EVENASPH | 6.5 | 0 | | 12 | 0 |
| 7 | EVENASPH | 6.5 | 3.05 | AQUE- | 12 | 0 |
| | | | | OUS | | |
| STO | STANDARD | 10.2 | −5e−008 | AQUE- | 3 | 0 |
| | | | | OUS | | |
| 9 | STANDARD | 10.2 | 4 | LENS | 8 | 0 |
| 10 | STANDARD | −6 | 0 | | 8 | 0 |
| 11 | STANDARD | −6 | 16.4804 | VITRE- | 0 | 0 |
| | | | | OUS | | |
| 12 | STANDARD | −11.7 | 0 | | 0 | 0 |
| IMA | STANDARD | −11.7 | 0 | VITRE- | 23.4 | 0 |
| | | | | OUS | | |

The exact values R1, R2, C1, and C2, of the two surfaces defining the wide field of view lens will vary depending on a selected merit function.

Table 3 provides index of refraction data for each of the surfaces of the lens system design.

TABLE 4
INDEX OF REFRACTIONDATA

| Sur- | | | Pres- | | | |
|---|---|---|---|---|---|---|
| face | Glass | Temp | sure | n1 | n2 | n3 |
| OBJ | | 20.00 | 1.00 | 1.00000000 | 1.00000000 | 1.00000000 |
| 1 | POLY- | 20.00 | 1.00 | 1.60407854 | 1.59048108 | 1.58494861 |
| | STR | | | | | |
| 2 | | 20.00 | 1.00 | 1.00000000 | 1.00000000 | 1.00000000 |
| 3 | | 20.00 | 1.00 | 1.00000000 | 1.00000000 | 1.00000000 |
| 4 | | 20.00 | 1.00 | 1.00000000 | 1.00000000 | 1.00000000 |
| 5 | COR- | 20.00 | 1.00 | 1.38069934 | 1.37710166 | 1.37405122 |
| | NEA | | | | | |
| 6 | | 20.00 | 1.00 | 1.00000000 | 1.00000000 | 1.00000000 |
| 7 | AQUE- | 20.00 | 1.00 | 1.34219136 | 1.33738065 | 1.33539337 |
| | OUS | | | | | |
| STO | AQUE- | 20.00 | 1.00 | 1.34219136 | 1.33738065 | 1.33539337 |
| | OUS | | | | | |
| 9 | LENS | 20.00 | 1.00 | 1.42623849 | 1.41997547 | 1.41749184 |
| 10 | | 20.00 | 1.00 | 1.00000000 | 1.00000000 | 1.00000000 |
| 11 | VITRE- | 20.00 | 1.00 | 1.34069139 | 1.33598142 | 1.33409377 |
| | OUS | | | | | |
| 12 | VITRE- | 20.00 | 1.00 | 1.34069139 | 1.33598142 | 1.33409377 |
| | OUS | | | | | |
| IMA | | 20.00 | 1.00 | 1.00000000 | 1.00000000 | 1.00000000 |

The present invention can be embodied in the form of computer-implemented processes and apparatus for practicing those processes. For example, the method of designing the lens could be automated to allow a lens to be customized according to the parameters of a specific eye. The present invention can also be embodied in the form of computer program code containing computer-readable instructions embodied in tangible media, such as floppy diskettes, CD-ROMs, hard drives, or any other computer-readable storage medium, wherein, when the computer program code is loaded into and executed by a computer, the computer becomes an apparatus for practicing the invention. The present invention can also be embodied in the form of computer program code, for example, whether stored in a storage medium, loaded into and/or executed by a computer, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose computer, the computer program code segments configure the computer to create specific logic circuits or processing modules.

While the preferred embodiments of the present invention have been shown and described herein, it will be obvious that such embodiments are provided by way of example only. Numerous variations, changes and substitutions will occur to those of skill in the art without departing from the invention herein. Accordingly, it is intended that the invention be limited only by the spirit and scope of the appended claims.

What is claimed is:

1. A lens system for use in eye surgery and diagnostics, in combination with a microscope, the system comprising a lens having a configuration for providing variable field curvature, wherein the concave surface of a fundus of the eye is in focus for at least the full extent of a retina of the eye.

2. The system of claim 1, wherein the lens is adapted to compensate for optical properties of eye components in an image space.

3. The system of claim 2, wherein the optical properties of eye components comprise the optical properties of a cornea, the optical properties of a crystalline lens, the optical properties of aqueous humor, and the optical properties of vitreous humor.

4. The system of claim 3, wherein the optical properties of each eye component comprise a surface, curvature, thickness, aperture, diameter, and refractive property.

6. The system of claim 1, wherein the lens is adapted to be placed in contact with a cornea of the eye.

7. The system of claim 1, wherein the lens is formed from a transparent polymer adapted for a one-time use.

8. The system of claim 1, wherein the lens is a two sided convex lens having a positive refractive power, wherein one or more of lens surfaces of the lens is aspherical.

9. A lens system for use in eye surgery and diagnostics, in combination with a microscope, the system comprising a lens having a configuration for providing a wide angle field of view, wherein the lens is adapted to compensate for optical properties of eye components in an image space.

10. The system of claim 9, wherein the lens further comprises a variable field curvature, wherein the concave surface of a fundus of the eye is in focus for substantially the full extent of a retina of the eye.

11. The system of claim 9, wherein the optical properties of eye components comprise the optical properties of a cornea, the optical properties of a crystalline lens, the optical properties of aqueous humor, and the optical properties of vitreous humor.

12. The system of claim 11, wherein the optical properties of each eye component comprise the surface, radius, thickness, aperture, diameter, and refractive properties of the respective eye component.

13. A method of designing a wide angle lens system for use in combination with a microscope for eye surgery and diagnostics, comprising:
defining a plurality of eye components having optical properties as surfaces in the lens system; and
calculating a wide angle lens design based on a desired field of view and the surfaces defined from the plurality of eye features modeled as optical components.

14. The method of claim 13, wherein the desired field of view is determined by providing a field curvature, so that the concave surface of a fundus of an eye is in focus for substantially the full extent of a retina of the eye.

15. The method of claim 13, wherein the optical properties of the eye components comprise the optical properties of a cornea, the optical properties of a crystalline lens, the optical properties of aqueous humor, and the optical properties of vitreous humor.

16. The method of claim 15, wherein the optical properties of each eye component comprise a surface, curvature, thickness, aperture, diameter, and refractive property.
